(19) United States
(12) Patent Application Publication
(10) Pub. No.: US 2006/0182702 A1
(43) Pub. Date: Aug. 17, 2006

(54) Composition containing a cation, a cationic polymer, a solid compound and a starch, and cosmetic treatment process

(75) Inventors: Pascale Lazzeri, Levallois Perret (FR); Laurent Chesneau, Levallois Perret (FR); Veronique Mahe, Vaux S/Seine (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/334,473

(22) Filed: Jan. 19, 2006

Related U.S. Application Data
(60) Provisional application No. 60/657,411, filed on Mar. 2, 2005.

(30) Foreign Application Priority Data
Feb. 11, 2005 (FR) 05 01414

(51) Int. Cl. A61K 8/73; A61K 8/81; A61K 8/40

(52) U.S. Cl. 424/70.13; 424/70.28

(57) ABSTRACT

The present invention relates to a composition containing, in a cosmetically acceptable aqueous medium, at least one cationic surfactant, at least one starch, preferably a modified starch, at least one non-silicone cationic polymer with a particular cationic charge and at least one nonionic non-polymeric solid compound with a melting point of greater than or equal to 35° C., and to a cosmetic process for treating keratin materials and in particular the hair. These compositions have an improved conditioning effect, especially smoothing of the ends.

### REFERENCE TO PRIOR APPLICATIONS

This application claims priority to U.S. provisional application 60/657,411 filed Mar. 2, 2005, and to French patent application 0501414 filed Feb. 11, 2005, both incorporated herein by reference.

### FIELD OF THE INVENTION

The present invention relates to a composition, especially a hair conditioner, comprising at least one cationic surfactant, at least one modified starch, at least one non-silicone cationic polymer and at least one nonionic non-polymeric solid compound, and to a process for treating keratin materials, in particular hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is well known that hair that has been sensitized (i.e. damaged and/or embrittled) to varying degrees under the action of atmospheric agents or under the action of mechanical or chemical treatments, such as dyeing, bleaching and/or permanent-waving, is often difficult to disentangle and to style, and lacks softness.

Cosmetic compositions comprising cationic surfactants and thickening polysaccharides especially such as starch or celluloses have already been proposed for treating keratin materials, and in particular the hair.

However, such compositions have drawbacks such as rinseability problems, stability problems, difficulties in distributing them over the keratin materials and also insufficient cosmetic properties.

It has already been recommended to use cationic polymers, cationic silicones or cationic surfactants in compositions for washing or caring for keratin materials such as the hair, to facilitate the disentangling of the hair and to give it softness and suppleness. The use of cationic polymers or cations for this purpose has various drawbacks. On account of their high affinity for the hair, some of these polymers become deposited in substantial amount during repeated use, and lead to undesirable effects such as an unpleasant, laden feel, stiffening of the hair, and adhesion between the fibres that affects styling.

Furthermore, the care used for very sensitized hair may be insufficient to treat the ends, which are usually very damaged.

In summary, it is found that the current conditioning cosmetic compositions are not entirely satisfactory. Thus, it is sought to obtain cosmetic compositions that have very good cosmetic properties, in particular on very sensitized hair.

### SUMMARY OF THE INVENTION

The inventors have now discovered that the combination of a cationic surfactant, a starch, preferably a modified starch, a non-silicone cationic polymer with a particular cationic charge, and a particular nonionic non-polymeric solid compound allows these drawbacks to be overcome.

Hair treated with this composition is smooth, disentangles easily, is shiny, supple and individualized, and has a soft feel with no feeling of residues. The hair has a natural, unladen appearance. The smoothness is uniform from the roots to the ends. The ends show less splitting.

Moreover, these effects are remanent over time.

### DETAILED DESCRIPTION OF THE PREFERRED EMBODIMENTS

According to the present invention novel compositions are now provided, comprising, preferably in a cosmetically acceptable aqueous medium, at least one cationic surfactant, at least one starch (preferably at least one modified starch), at least one non-silicone cationic polymer with a cationic charge density of greater than or equal to 5 meq./g and at least one nonionic non-polymeric solid compound with a melting point of greater than or equal to 35° C. and/or with a viscosity, at a temperature of 40° C. and at a shear rate of 1 s⁻¹, of greater than or equal to 1 Pa·s.

Another subject of the invention is a cosmetic process for treating keratin materials, in particular the hair, using the abovementioned composition.

Another subject of the invention is the use of the invention composition as a hair conditioner.

Other subjects, characteristics, aspects and advantages of the invention will emerge even more clearly on reading the further description and the various examples that follow.

According to the present invention, the term “sensitized hair” means hair that has undergone external physical attack (by light, heat, waves, etc.), mechanical attack (by repeated blow-drying, combing or brushing, etc.) or chemical attack (by oxidation dyeing, bleaching, permanent-waving, relaxing, etc.), or any combination thereof. Among these types of attack, the particularly deleterious nature of chemical attack will be noted. The compositions according to the invention are particularly effective on hair sensitized by chemical attack.

The term “at least one” will be understood as meaning “one or more”, i.e. one, two, three or more.

The term “cosmetically acceptable medium” means a medium that is compatible with any keratin material, such as the skin, the hair, the nails, the eyelashes, the eyebrows or the lips and any other area of body and facial skin.

The composition according to the invention comprises one or more cationic surfactants. Examples of such surfactants are well known, such as optionally polyoxyalkylenated primary, secondary or tertiary fatty amine salts and quaternary ammonium salts, and mixtures thereof.

According to the invention, the cationic surfactants are preferably non-polymeric.

Examples of quaternary ammonium salts that may especially be mentioned include:

those of general formula (V) below:

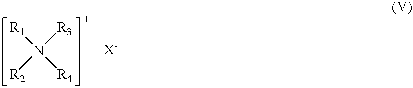

in which the symbols R₁ to R₄, which may be identical or different, represent a linear or branched aliphatic radical containing from 1 to 30 carbon atoms, or an aromatic radical such as aryl or alkylaryl. In a preferred embodiment at least one R1 to R4 represent a linear or branched aliphatic radical comprising from 8 to 30 carbon atoms. The aliphatic radicals may comprise hetero atoms such as, especially, oxygen, nitrogen, sulfur and halogens. The aliphatic radicals are chosen, for example, from

(C₁-C₃₀)alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals, containing from about 1 to 30 carbon atoms; X⁻ is an anion chosen from the group of halides, phosphates, acetates, lactates, (C₂-C₆) alkyl sulfates and alkyl- or alkylaryl-sulfonates;

quaternary ammonium salts of imidazoline, for instance those of formula (VI) below: 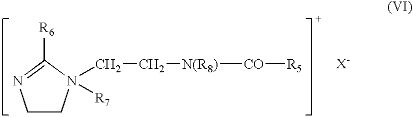
in which R₅ represents an alkenyl or alkyl radical containing from 8 to 30 carbon atoms, for example fatty acid derivatives of tallow or of coconut, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, and X is an anion chosen from the group of halides, phosphates, acetates, lactates, alkyl sulfates, alkyl sulfonates or alkylaryl sulfonates. R₅ and R₆ preferably denote a mixture of alkenyl or alkyl radicals containing from 12 to 21 carbon atoms, such as, for example, fatty acid derivatives of tallow, R₇ denotes methyl and R₈ denotes hydrogen. Such a product is, for example, Quaternium-27 (CTFA 2002), Quaternium-87 (CTFA 2002) or Quaternium-83 (CTFA 2002), which are sold under the name “Varisoft®” W575PG by the company Goldschmidt,

diquaternary ammonium salts of formula (VII):

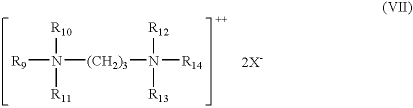

in which R₉ denotes an aliphatic radical containing from about 16 to 30 carbon atoms, R₁₀, R₁₁, R₁₂, R₁₃ and R₁₄ which may be identical or different, are chosen from hydrogen and an alkyl radical containing from 1 to 4 carbon atoms, and X⁻ is an anion chosen from the group of halides, acetates, phosphates, nitrates, ethyl sulfates and methyl sulfates. Such diquaternary ammonium salts in particular comprise propanetallow-diammonium dichloride;

quaternary ammonium salts containing at least one ester function, such as those of formula (VIII) below:

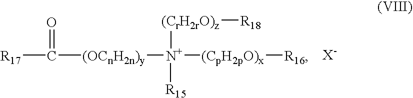

in which:

R₁₅ is chosen from C₁-C₆ alkyl radicals and C₁-C₆ hydroxyalkyl or dihydroxyalkyl radicals;

R₁₆ is chosen from:

a radical

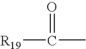

linear or branched, saturated or unsaturated C₁-C₂₂ hydrocarbon-based radicals R₂₀,

a hydrogen atom,

R₁₇ is chosen from:

a radical

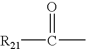

linear or branched, saturated or unsaturated C₁-C₆ hydrocarbon-based radicals R₂₂,

a hydrogen atom,

R₁₇, R₁₉ and R₂₁, which may be identical or different, are chosen from linear or branched, saturated or unsaturated C₇-C₂₁ hydrocarbon-based radicals;

r, n and p, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₁₆ denotes R₂₀ and that when z is 0, then R₁₈ denotes R₂₂.

The alkyl radicals R₁₅ may be linear or branched, and more particularly linear.

Preferably, R₁₅ denotes a methyl, ethyl, hydroxyethyl or dihydroxypropyl radical, and more particularly a methyl or ethyl radical.

Advantageously, the sum x+y+z is from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

Advantageously, R₁₇, R₁₉ and R₂₁, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are 0 or 1.

Advantageously, y is equal to 1.

Preferably, r, n and p, which may be identical or different, are equal to 2 or 3 and even more particularly equal to 2.

The anion X⁻ is preferably a halide (chloride, bromide or iodide) or a C₁-C₄ alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function may be used.

The anion X⁻ is even more particularly chloride or methyl sulfate.

Use is made more particularly in the composition according to the invention of the ammonium salts of formula (IV) in which:

R₁₅ denotes a methyl or ethyl radical,

x and y are equal to 1;

z is equal to 0 or 1;

r, n and p are equal to 2;

R₁₆ is chosen from:

a radical

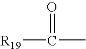

methyl, ethyl or C₁₄-C₂₂ hydrocarbon-based radicals,

a hydrogen atom;

R₁₈ is chosen from:

a radical

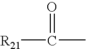

a hydrogen atom;

R₁₇, R₁₉ and R₂₁, which may be identical or different, are chosen from linear or branched, saturated or unsaturated C₁₃-C₁₇ hydrocarbon-based radicals, and preferably from linear or branched, saturated or unsaturated C₁₃-C₁₇ alkyl and alkenyl radicals.

The hydrocarbon-based radicals are advantageously linear.

Examples of compounds of formula (VIII) that may be mentioned include the salts (especially chloride or methyl sulfate) of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

These products are obtained, for example, by direct esterification of optionally oxyalkylenated triethanolamine, triisopropanolamine, alkyldiethanol-amine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

The composition according to the invention may preferably contain a mixture of quaternary ammonium mono-, di- and triester salts with a weight majority of diester salts.

Examples of mixtures of ammonium salts that may be used include the mixture containing 15% to 30% by weight of acyloxyethyl-dihydroxyethyl-methylammonium methyl sulfate, 45% to 60% of diacyloxyethyl-hydroxyethyl-methylammonium methyl sulfate and 15% to 30% of triacyloxyethyl-methylammonium methyl sulfate, the acyl radicals containing from 14 to 18 carbon atoms and being derived from optionally partially hydrogenated palm oil.

It is also possible to use the ammonium salts containing at least one ester function described in patents U.S. Pat. No. 4,874,554 and U.S. Pat. No. 4,137,180.

Among the quaternary ammonium salts mentioned above that are preferably used are those corresponding to formula (V). Mention may especially be made firstly of tetraalkylammonium chlorides, for instance dialkyldimethylammonium or alkyltrimethylammonium chlorides in which the alkyl radical contains from about 12 to 22 carbon atoms, in particular behenyltrimethylammonium, distearyldimethylammonium, cetyltrimethylammonium or benzyldimethylstearylammonium chlorides, or alternatively palmitylamidopropyl-trimethylammonium chloride or stearamidopropyldimethyl-(myristyl acetate)ammonium chloride corresponding to Quaternium-70 (CTFA 2002) sold under the name Ceraphyl® 70 by the company ISP.

The cationic surfactants that are particularly preferred in the composition of the invention are chosen from quaternary ammonium salts, and in particular from behenyltrimethylammonium chloride, cetyltrimethylammonium chloride, Quaternium-83, Quaternium-87, behenylamidopropyl-2,3-dihydroxypropyldimethylammonium chloride and palmitylamidopropyltrimethylammonium chloride.

The composition according to the invention preferably comprises the cationic surfactant(s) in any amount, including in an amount ranging from 0.05% to 10% by weight, preferably from 0.1% to 8% by weight and more particularly from 0.2% to 5% by weight relative to the total weight of the composition.

The cosmetic composition according to the invention comprises one or more cationic polymers whose cationic charge density is greater than or equal to 5 milliequivalents per gram (meq./g) and preferably ranging from 5 to 20 meq./g.

The cationic charge density of a polymer corresponds to the number of moles of cationic charges per unit of mass of polymer under conditions in which this polymer is totally ionized. It may be determined by calculation if the structure of the polymer is known, i.e. the structure of the monomers constituting the polymer and their molar or weight proportion. It may also be determined experimentally via the Kjeldahl method.

The cationic polymers with a cationic charge density of greater than or equal to 5 meq./g that may be used in accordance with the present invention are not limited and may be chosen from all those already known, especially those known for improving the cosmetic properties of the hair treated with compositions, i.e. especially those described in patent application EP-A-0 337 354 and in French patent applications FR-A-2 270 846, 2 383 660, 2 598 611, 2 470 596 and 2 519 863.

In general, for the purposes of the present invention, the term “cationic polymer” denotes any polymer comprising cationic groups and/or groups that may be ionized into cationic groups.

The cationic polymers may be chosen from those containing units comprising primary, secondary, tertiary and/or quaternary amine groups that either may form part of the main polymer chain or may be borne by a side substituent directly attached thereto.

The cationic polymers used preferably have a number-average molecular mass of between 500 and 5×10⁶ approximately and preferably between 10³ and 3×10⁶ approximately.

Among the cationic polymers that may be mentioned more particularly are polymers of the polyamine, polyamino amide and polyquaternary ammonium type including those that are known products.

Examples of the polymers of the polyamine, polyamino amide and polyquaternary ammonium type that may be used in accordance with the present invention, and that may especially be mentioned, are those described in French patents 2 505 348 and 2 542 997. Among these polymers, mention may be made of:

(1) quaternized or non-quaternized vinylpyrrolidone/dialkylaminoalkyl (meth)acrylate copolymers,

(2) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers are described, in particular, in French patents 2 162 025 and 2 280 361;

(3) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508;

(4) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl group contains from 1 to 4 carbon atoms and preferably denotes methyl, ethyl or propyl. Such polymers are described in particular in French patent 1 583 363.

(5) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid being between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

(6) cyclopolymers of alkyldiallylamine or of dialkyldiallylammonium, such as the homopolymers or copolymers containing units corresponding to formula (VI) or (VI′):

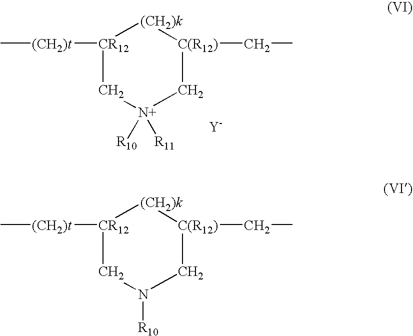

in which formulae k and t are equal to 0 or 1, the sum k+t being equal to 1; R₁₂ denotes a hydrogen atom or a methyl group; R₁₀ and R₁₁, independently of each other, denote an alkyl group containing from 1 to 6 carbon atoms, a hydroxyalkyl group in which the alkyl group preferably contains 1 to 5 carbon atoms, a lower amidoalkyl group, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406.

Mention may be made, for example, of the diallyldimethylammonium chloride homopolymer sold under the name “Merquat® 100” by the company Ondeo-Nalco, and copolymers of diallyldimethylammonium chloride and of acrylamide.

(7) a diquaternary ammonium polycondensate containing repeating units corresponding to the formula:

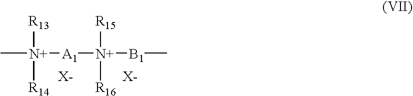

in which formula (VII):

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic groups, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl group substituted with a nitrile, ester, acyl or amide group or a group —CO—O—R₁₇-D or —CO—NH—R₁₇-D where R₁₇ is an alkylene group and D is a quaternary ammonium group;

A₁ and B₁ represent polymethylene groups containing from 2 to 20 carbon atoms, which groups may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from an inorganic or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene group, B₁ can also denote a group —(CH₂)ₙ—CO-D-OC— (CH₂)ₙ— in which D denotes:

a) a glycol residue of formula: —O-Z-O—, where Z denotes a linear or branched hydrocarbon-based group or a group corresponding to one of the following formulae:
—(CH₂—CH₂—O)ₓ—CH₂—CH₂—
—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based group, or alternatively the divalent radical
—CH₂—CH₂—S—S—CH₂—CH₂—;

d) a ureylene group of formula: —NH—CO—NH—;

Preferably, X⁻ is an anion such as chloride or bromide.

These polymers generally have a number-average molecular mass of between 1 000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to the formula:

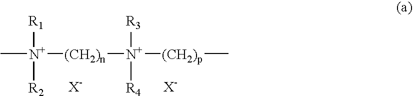

in which R₁, R₂, R₃ and R₄, which may be identical or different, denote an alkyl or hydroxyalkyl group containing from 1 to 4 carbon atoms approximately, n and p are integers ranging from 2 to 20 approximately, and X⁻ is an anion derived from an inorganic or organic acid.

One compound of formula (a) which is particularly preferred is the one for which R₁, R₂, R₃ and R₄ represent a methyl group and n=3, p=6 and X═Cl, which is known as Hexadimethrine chloride according to the INCI (CTFA) nomenclature.

(8) polyquaternary ammonium polycondensates consisting of units of formula (VIII):

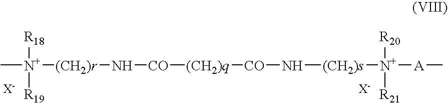

in which formula:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂ (OCH₂CH₂)ₚOH group,

where p is equal to 0 or to an integer between 1 and 6, with the proviso that R₁₈, R₁₉, R₂₀ and R₂₁ do not simultaneously represent a hydrogen atom,

r and s, which may be identical or different, are integers between 1 and 6,

q is equal to 0 or to an integer between 1 and 34,

X denotes a halogen atom,

A denotes a dihalide group or preferably represents —CH₂—CH₂—O—CH₂—CH₂—.

Such compounds are described in particular in patent application EP-A-122 324.

Among these products, mention may be made, for example, of the products “Mirapol® A 15”, “Mirapol® AD1”, “Mirapol® AZ1” and “Mirapol® 175” sold by the company Miranol.

(9) homopolymers or copolymers derived from acrylic or methacrylic acids and comprising units:

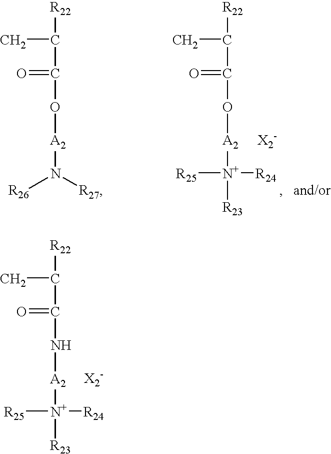

in which:

the groups R₂₂ independently denote H or CH₃,

the groups A₂ independently denote a linear or branched alkyl group of 1 to 6 carbon atoms or a hydroxyalkyl group of 1 to 4 carbon atoms,

the groups R₂₃, R₂₄ and R₂₅, which may be identical or different, independently denote an alkyl group of 1 to 18 carbon atoms, or a benzyl group,

the groups R₂₆ and R₂₇ represent a hydrogen atom or an alkyl group of 1 to 6 carbon atoms,

X₂⁻ denotes an anion, for example methosulfate or halide such as chloride or bromide.

The comonomer(s) that may be used in the preparation of the corresponding copolymers belong(s) to the family of acrylamides, methacrylamides, diacetone acrylamides, acrylamides and methacrylamides substituted on the nitrogen with lower alkyls, alkyl esters, acrylic or methacrylic acids, vinylpyrrolidone or vinyl esters.

(10) Quaternary polymers of vinylpyrrolidone and of vinylimidazole.

(11) Crosslinked methacryloyloxy(C₁-C₄)alkyl-tri(C₁-C₄)alkylammonium salt polymers such as the polymers obtained by homopolymerization of dimethyl-aminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide.

Other cationic polymers that can be used in the context of the invention include polyalkyleneimines, in particular polyethyleneimines, polymers containing vinylpyridine or vinylpyridinium units, condensates of polyamines and of epichlorohydrin, quaternary polyureylenes and chitin derivatives.

Among all the cationic polymers that may be used in the context of the present invention, it is preferred to use dialkyldiallylammonium halide homopolymers and copolymers, polyethyleneimines and polycondensates containing diquaternary ammonium or polyquaternary ammonium repeating units.

The cationic polymers described above are preferably present in an amount ranging from 0.01% to 10% by weight, better still from 0.05% to 5% by weight and even more preferentially from 0.1% to 3% by weight relative to the total weight of the composition.

The starches that may be used in the present invention are more particularly macromolecules in the form of polymers consisting of elementary units, which are anhydroglucose units. The number of these units and their assembly make it possible to distinguish amylose (linear polymer) and amylopectin (branched polymer). The relative proportions of amylose and of amylopectin, and also their degree of polymerization, vary as a function of the botanical origin of the starches.

The botanical origin of the starch molecules used in the present invention is not limited and may be cereals or tubers. Thus, the starches are chosen, for example, from corn starch, rice starch, cassava starch, barley starch, potato starch, wheat starch, sorghum starch and pea starch.

The starches used in the composition of the invention are preferably modified. The starches may be chemically or physically modified: especially via one or more of the following reactions: pregelatinization, oxidation, crosslinking, esterification, etherification, amidation, heat treatments.

More particularly, these reactions may be performed in the following manner:

pregelatinization by splitting the starch granules (for example drying and cooking in a drying drum);

oxidation with strong oxidizing agents, leading to the introduction of carboxyl groups into the starch molecule and to depolymerization of the starch molecule (for example by treating an aqueous starch solution with sodium hypochlorite);

crosslinking with functional agents capable of reacting with the hydroxyl groups of the starch molecules, which will thus bond together (for example with glyceryl and/or phosphate groups);

esterification in alkaline medium for the grafting of functional groups, especially C₁-C₆ acyl (acetyl), C₁-C₆ hydroxyalkyl (hydroxyethyl or hydroxypropyl), carboxymethyl or octenylsuccinic.

Distarch phosphates (of the type Am—O—PO—(OX)—O—Am) or even tristarch phosphates (of the type Am—O—PO—(O—Am)₂) or mixtures thereof may especially be obtained by crosslinking with phosphorus compounds of monostarch phosphates (of the type Am—O—PO—(OX)₂).

X especially denotes alkali metals (for example sodium or potassium), alkaline-earth metals (for example calcium or magnesium), ammonium salts, amine salts, for instance those of monoethanolamine, diethanolamine, triethanolamine, 3-amino-1,2-propane-diol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Distarch phosphates or compounds rich in distarch phosphate will preferentially be used, for instance the products sold under the references Pregel VA-70-T AGGL (gelatinized hydroxypropyl cassava distarch phosphate), Pregel TK1 (gelatinized cassava distarch phosphate) and Pregel 200 (gelatinized acetyl cassava distarch phosphate) by the company Avebe, or Structure Zea from National Starch (gelatinized corn distarch phosphate).

A preferred starch is a starch that has undergone at least one chemical modification such as at least one esterification.

According to the invention, amphoteric starches may also be used, these amphoteric starches comprising one or more anionic groups and one or more cationic groups. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The amphoteric starches are especially chosen from the compounds having the following formulae:

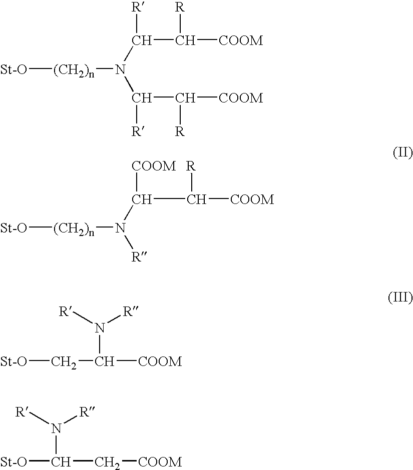

in which formulae:

St-O represents a starch molecule,

R, which may be identical or different, represents a hydrogen atom or a methyl radical,

R′, which may be identical or different, represents a hydrogen atom, a methyl radical or a —COOH group,

n is an integer equal to 2 or 3,

M, which may be identical or different, denotes a hydrogen atom, an alkali metal or alkaline-earth metal such as Na, K, Li or NH₄, a quaternary ammonium or an organic amine,

R″ represents a hydrogen atom or an alkyl radical containing from 1 to 18 carbon atoms.

These compounds are especially described in patents U.S. Pat. No. 5,455,340 and U.S. Pat. No. 4,017,460, which are included by way of reference.

The starch molecules may for example be derived from any plant source of starch, such as, especially, corn, potato, oat, rice, tapioca, sorghum, barley or wheat. Hydrolysates of the starches mentioned above may also be used. The starch is preferably derived from potato.

The starches of formula (I) or (II) are particularly used. The starches modified with 2-chloro-ethylaminodipropionic acid are more particularly used, i.e. the starches of formula (I) or (II) in which R, R′, R″ and M represent a hydrogen atom and n is equal to 2. The preferred amphoteric starch is a starch chloroethylamidodipropionate.

According to the invention, the preferably modified starch(es) may represent from 0.1% to 20% by weight, preferably from 0.5% to 15% by weight and more particularly from 1% to 10% by weight relative to the total weight of the final composition.

The solid compounds according to the invention may be crystalline, amorphous or pasty.

The melting point preferably ranges from 35 to 250° C. and more particularly from 40 to 150° C.

These solids have a viscosity, at a temperature of 40° C. and at a shear rate of 1 s⁻¹, ranging from 1 Pa·s to 1 000 000 Pa·s and preferably from 10 to 1000 Pa·s.

The viscosity measurements may be taken at a temperature of about 40° C., using a Carri-Med CSL2-500 viscometer.

The melting point may be measured by DSC or on a Köfler bench. The melting point may be measured by differential calorimetric analysis (DSC) with a temperature increase rate of 10° C./minute. The melting point is then the temperature corresponding to the top of the endothermic melting peak obtained during the measurement.

The nonionic non-polymeric solid compounds with a melting point of greater than or equal to 35° C. are especially chosen from oxyethylenated or non-oxyethylenated fatty alcohols, fatty esters, mineral waxes and organic waxes other than fatty esters and fatty alcohols, and mixtures thereof.

The fatty alcohols according to the invention are preferably linear and saturated, and contain from 12 to 40 carbon atoms.

The fatty alcohols preferably have the structure R—OH, in which R preferably denotes a C₁₂-C₂₄ alkyl group. R may be substituted with one or more hydroxyl groups.

Examples that may be mentioned include myristyl alcohol, cetyl alcohol, stearyl alcohol and behenyl alcohol, and mixtures thereof.

The fatty alcohol may represent a mixture of fatty alcohols, which means that, in a commercial product, several fatty alcohol species may coexist in the form of a mixture.

Preferably, the fatty alcohols of the invention are non-oxyalkylenated and/or non-glycerolated. These fatty alcohols may be constituents of animal or plant waxes.

The fatty esters are fatty acid esters, i.e. esters of a carboxylic acid containing at least 10 carbon atoms and of a monoalcohol or a polyol. The fatty esters according to the invention may be monoesters, diesters or triesters.

The carboxylic acids preferably contain from 10 to 30 carbon atoms and more particularly from 12 to 24 carbon atoms. The alcohols preferably contain from 10 to 30 carbon atoms and more particularly from 12 to 24 carbon atoms. Preferably, the solid fatty esters according to the invention are esters of a monocarboxylic fatty acid containing at least 10 carbon atoms and of a monoalcohol containing at least 10 carbon atoms.

Esters according to the invention that may be mentioned include cetyl myristate and myristyl myristate.

The fatty esters may be constituents of animal or plant waxes.

For the purposes of the present invention, a wax is a lipophilic compound that is solid at room temperature (about 25° C.), with a reversible solid/liquid change of state, having a melting point of greater than about 40° C., which may be up to 200° C., and having anisotropic crystal organization in the solid state. In general, the size of the wax crystals is such that the crystals diffract and/or scatter light, giving the composition comprising them a more or less opaque hazy appearance. By bringing the wax to its melting point, it is possible to make it miscible with oils and to form a microscopically uniform mixture, but on returning the temperature of the mixture to room temperature, recrystallization of the wax in the oils of the mixture is obtained, which is detectable microscopically and macroscopically (opalescence).

As waxes that may be used in the present invention, mention may especially be made of waxes of plant origin such as beeswax, spermaceti, lanolin wax and lanolin derivatives; plant waxes such as carnauba wax, candelilla wax, ouricury wax, Japan wax, cocoa butter wax, cork fibre wax or sugarcane wax; mineral waxes, for example paraffin wax, petroleum jelly wax, lignite wax or microcrystalline waxes, ozokerites, olive wax, rice wax, hydrogenated jojoba wax or the absolute waxes of flowers, such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and mixtures thereof. Organic waxes that may also be mentioned include waxes comprising amide functions and in particular natural or synthetic ceramides.

For a definition of waxes, mention may be made, for example, of P.D. Dorgan, Drug and Cosmetic Industry, December 1983, pp. 30-33.

The wax(es) is (are) chosen especially from carnauba wax, candelilla wax, esparto grass wax, paraffin wax, ozokerite, plant waxes, for instance olive wax, rice wax, hydrogenated jojoba wax or the absolute waxes of flowers, such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes, such as the product sold by the company Sophim under the reference M82.

The solid compound(s) may be present in the composition in a content ranging from 0.1% to 15%, preferably from 0.5% to 10% and more preferably from 1% to 8% by weight relative to the total weight of the composition.

The composition according to the invention may optionally contain surfactants other than cationic surfactants.

The surfactants may be present in, for example, an amount ranging from 0.1% to 10%, preferably from 0.5% to 8% and even more preferably from 1% to 5% by weight approximately, relative to the total weight of the composition.

The additional surfactants are preferably chosen from nonionic surfactants.

Nonionic surfactants are compounds that are well known per se (see in particular in this respect “Handbook of Surfactants” by M. R. Porter, published by Blackie & Son (Glasgow and London), 1991, pp. 116-178) and, in the context of the present invention, their nature is not a critical feature. Thus, they can be chosen in particular from (non-limiting list) polyethoxylated, polypropoxylated or polyglycerolated fatty alcohols, alkylphenols, α-diols or acids having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

The compositions according to the invention are preferably non-washing (non-detergent) compositions, and preferably comprise less than 4% by weight and more particularly less than 1% by weight of anionic surfactants relative to the total weight of the composition.

The composition according to the invention may also comprise at least one additional conditioning agent chosen from silicones, cationic polymers other than the cationic polymers according to the invention, carboxylic fatty esters other than those of the invention, plant oils, mineral oils and synthetic oils such as poly(α-olefins), and mixtures thereof.

The silicones that may be used in accordance with the invention may be soluble or insoluble in the composition, and they may be in particular polyorgano-siloxanes that are insoluble in the composition of the invention. They may be in the form of oils, waxes, resins or gums. They may be used pure or as an emulsion, a dispersion or a microemulsion.

The organopolysiloxanes are defined in greater detail in Walter Noll's “Chemistry and Technology of Silicones” (1968) Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5” by Rhodia, and mixtures thereof.

Mention may also be made of cyclocopolymers of the dimethylsiloxane/methylalkylsiloxane type, such as “Silicone Volatile FZ 3109” sold by the company Union Carbide, having the chemical structure:

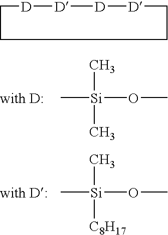

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Among the non-volatile silicones that may especially be mentioned are polyalkylsiloxanes, polyarylsiloxanes, polyalkylarylsiloxanes, silicone gums and resins, polyorganosiloxanes modified with organofunctional groups, and also mixtures thereof.

The organomodified silicones that can be used in accordance with the invention are silicones as defined above and containing in their structure one or more organofunctional groups attached via a hydrocarbon-based group.

Among the organomodified silicones, mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes containing a hydroxyalkyl function, described in French patent application FR-A-85/16334;

acyloxyalkyl groups such as, for example, the polyorganosiloxanes described in U.S. Pat. No. 4,957,732;

anionic groups of the carboxylic acid type, such as, for example, in the products described in patent EP 186 507 from the company Chisso Corporation, or of the alkylcarboxylic type, such as those present in the product X-22-3701^{E} from the company Shin-Etsu; 2-hydroxyalkyl sulfonate; 2-hydroxyalkyl thiosulfate such as the products sold by the company Goldschmidt under the names “Abil® S201” and “Abil® S255”;

hydroxyacylamino groups, such as the polyorganosiloxanes described in patent application EP 342 834. Mention may be made, for example, of the product Q2-8413 from the company Dow Corning.

Examples of silicones that are preferably used include polydimethylsiloxanes, polyalkylarylsiloxanes and polydimethylsiloxanes containing amino or alkoxylated groups.

The composition according to the invention may also comprise one or more liquid carboxylic acid esters, for instance purcellin oil (stearyl octanoate), isopropyl myristate, isopropyl palmitate, butyl stearate, hexyl laurate, diisopropyl adipate, isononyl isononanoate, 2-ethylhexyl palmitate, 2-octyldodecyl lactate, isostearyl neopentanoate, tridecyl neopentanoate, isocetyl neopentanoate and isoarachidyl neopentanoate, and mixtures thereof.

The composition according to the invention may also comprise one or more plant oils such as sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, maize oil, hazelnut oil, shea butter, palm oil, apricot kernel oil and beauty-leaf oil, and mixtures thereof.

Mineral oils that may especially be mentioned include liquid paraffin and liquid petroleum jelly.

The additional conditioning agents are preferably contained in the composition according to the invention in an amount ranging from 0.01% to 20% by weight, better still ranging from 0.1% to 10% by weight and more particularly ranging from 0.3% to 5% by weight relative to the total weight of the composition.

The cosmetically acceptable medium is preferably aqueous and may comprise water or a mixture of water and a cosmetically acceptable solvent such as a C₁-C₄ lower alcohol, for example ethanol, isopropanol, tert-butanol or n-butanol; polyols, for instance propylene glycol or glycerol; polyol ethers; C₅-C₁₀ alkanes; and mixtures thereof. The solvents are preferably chosen from glycerol and propylene glycol.

The cosmetically acceptable medium, which is especially aqueous, represents from 30% to 98% by weight relative to the total weight of the composition.

The solvents are preferably present in concentrations ranging from 0.5% to 30% by weight relative to the total weight of the composition.

The pH of the compositions of the invention is preferably between 2 and 8, and more preferably between 3 and 7.

The compositions according to the invention may also contain other additives including those that are well known in the art, such as anionic, nonionic or amphoteric polymers, non-polymeric thickeners, for instance acids or electrolytes, opacifiers, nacreous agents, vitamins, provitamins such as panthenol, fragrances, dyes, organic or mineral particles, preserving agents and pH stabilizers.

A person skilled in the art will take care to select the optional additives and the amount thereof such that they do not harm the properties of the compositions of the present invention.

These additives are present in the composition according to the invention in an amount ranging, for example, from 0% to 20% by weight relative to the total weight of the composition.

The compositions of the invention may be in any form, including in the form of a rinse-out or leave-in conditioner, permanent waving, relaxing, dyeing or bleaching compositions, or alternatively in the form of rinse-out compositions to be applied before or after a dyeing, bleaching, permanent-waving or relaxing operation or alternatively between the two steps of a permanent-waving or relaxing operation.

They may be used, for example, as hair conditioners, care products, deep-down care masks or scalp treatment lotions or creams. These compositions may be rinse-out or leave-in compositions.

According to one preferred embodiment of the invention, the composition may be used as a hair conditioner, in particular on sensitized hair. This hair conditioner may be a rinse-out or leave-in hair conditioner, preferably a rinse-out hair conditioner.

The compositions according to the invention may be in the form of a gel, a milk, a cream, an emulsion, fluid or thickened lotions or a foam, and may be used for the skin, the nails, the eyelashes, the lips and, more particularly, the hair.

The compositions may be packaged in any of various forms, especially in vaporizers, pump-dispenser bottles or in aerosol containers in order to dispense the composition in vaporized form or in the form of a mousse. Such packaging forms are indicated, for example, when it is desired to obtain a spray, a lacquer or a mousse for treating the hair.

The present invention also relates to a cosmetic process for treating keratin materials such as, for example, the skin or the hair, which consists in applying an effective amount of a cosmetic composition as described above to the keratin materials, and optionally rinsing it off after optionally leaving it to act for a period of time.

The rinsing is performed, for example, with water.

Thus, this process according to the invention allows the treatment, conditioning and care of the hair or any other keratin material.

The examples that follow illustrate the present invention and should not in any way be considered as limiting the invention.

### EXAMPLES 1 TO 3

The following rinse-out hair-conditioning compositions were prepared:

| | 1 | 2 | 3 |
|---|---|---|---|
| Stearyl alcohol | 2 g | 2 g | — |
| Myristyl alcohol | — | — | 2 g |
| N-Oleoyldihydrosphingosine | 0.4 g | 0.4 g | 0.4 g |
| Behenyltrimethylammonium chloride as an | 0.5 g | 0.5 g | — |
| aqueous solution containing 80% AM | (0.4 g AM) | (0.4 g AM) | |
| (Genamin KDMP from Clariant) | | | |
| Quaternium-87 (Varisoft W575PG from | 3.35 g | 3.35 g | 3.35 g |
| Goldschmidt) | (2.5 g AM) | (2.5 g AM) | (2.5 g AM) |
| Cetyltrimethylammonium chloride as an | — | — | 1 g |
| aqueous solution containing 25% active | | | (0.25 g AM) |
| material (Arquad 16-25 LO from Akzo | | | |
| Nobel) | | | |
| Poly (dimethyldiallylammonium chloride) | — | 0.63 g | — |
| as an aqueous solution containing 40% | | (0.252 g AM) | |
| active material | | | |
| charge density = 6.2 mEq./g (Merquat ® | | | |
| 100 from Ondeo-Nalco) | | | |
| Tetramethylhexamethylenediamine | 0.42 g | 0.42 g | 0.42 g |
| polycondensate as an aqueous solution | (0.252 g AM) | (0.252 g AM) | (0.252 g AM) |
| containing 60% AM (Mexomer PO from | | | |
| Chimex) | | | |
| Polydimethylsiloxane containing amino- | 5.9 g | — | — |
| methyl iminopropyl groups, as a | (0.885 g AM) | | |
| nonionic aqueous emulsion containing | | | |
| 15% AM (Belsil ADM 8020 P from Wacker) | | | |
| Polydimethylsiloxane (DC200 Fluid — | — | — | 1 g |
| 60 000 cs from Dow Corning) | | | |
| Pregelatinized hydroxypropyl corn | 5 g | | 5 g |
| distarch phosphate (Structure Zea from | | | |
| National Starch) | | | |
| Potato starch modified with 2-chloro- | — | 1 g | — |
| ethylaminodipropionic acid neutralized | | | |
| with sodium hydroxide (Structure | | | |
| Solanace from National Starch) | | | |
| Fragrance, preserving agents | Qs | qs | qs |
| Water qs | 100 g | 100 g | 100 g |

These compositions were applied to very sensitized hair. The cosmetic properties (disentangling, smoothness and suppleness) obtained were excellent and uniform from the roots to the ends of the hair. The ends were not split.

Between two applications, the hair remains soft, supple and smooth.

The above written description of the invention provides a manner and process of making and using it such that any person skilled in this art is enabled to make and use the same, this enablement being provided in particular for the subject matter of the appended claims, which make up a part of the original description and including a cosmetic composition comprising, in a cosmetically acceptable medium, at least one cationic surfactant, at least one starch, at least one non-silicone cationic polymer with a cationic charge density of greater than or equal to 5 meq./g and at least one nonionic non-polymeric solid compound with a melting point of greater than or equal to 35° C. and/or with a viscosity, at a temperature of 40° C. and at a shear rate of 1 s⁻¹, of greater than or equal to 1 Pa·s.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

Where a certain polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted “monomers,” but instead is made up of repeating units derived from reacted monomers.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a cosmetically acceptable medium,
at least one cationic surfactant,
at least one starch,
at least one non-silicone cationic polymer with a cationic charge density of greater than or equal to 5 meq./g, and
at least one nonionic non-polymeric solid compound with a melting point of greater than or equal to 35° C. and/or with a viscosity, at a temperature of 40° C. and at a shear rate of 1 s⁻¹, of greater than or equal to 1 Pa·s.

2. The composition according to claim 1, wherein the at least one cationic surfactant is/are chosen from optionally polyoxyalkylenated primary, secondary or tertiary fatty amine salts and quaternary ammonium salts, and mixtures thereof.

3. The composition according to claim 2, wherein said composition comprises at least one quaternary ammonium salt, and wherein the quaternary ammonium salt(s) are chosen from:
those having the general formula (V) below: 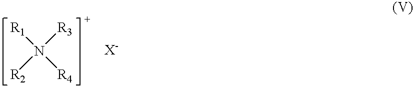
in which the symbols R₁ to R₄, which may be identical or different, represent a linear or branched aliphatic radical containing from 1 to 30 carbon atoms or an aromatic radical but at least one R1 to R4 represent a linear or branched aliphatic radical comprising from 8 to 30 carbon atoms; X⁻ is an anion chosen from the group of halides, phosphates, acetates, lactates, (C₂-C₆)alkyl sulfates, alkylsulfonates and alkylarylsulfonates;
quaternary ammonium salts of imidazoline;
diquaternary ammonium salts of formula (VII): 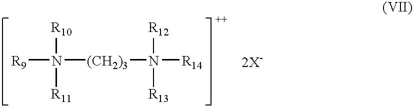
in which R₉ denotes an aliphatic radical containing from about 16 to 30 carbon atoms, R₁₀, R₁₁, R₁₂, R₁₃ and R₁₄, which may be identical or different, are chosen from hydrogen and an alkyl radical with from 1 to 4 carbon atoms, and X⁻ is an anion chosen from the group of halides, acetates, phosphates, nitrates and methyl sulfates;
quaternary ammonium salts comprising at least one ester function.

4. The composition according to claim 3, comprising at least one quaternary ammonium salt chosen from those of formula (VI) below: 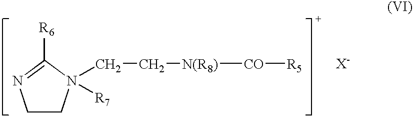
in which R₅ represents an alkenyl or alkyl radical containing from 8 to 30 carbon atoms, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, X⁻ is an anion chosen from the group of halides, phosphates, acetates, lactates, alkyl sulfates, alkylsulfonates and alkylarylsulfonates.

5. The composition according to claim 1, comprising at least one cationic surfactants chosen from behenyltrimethylammonium chloride, cetyltrimethylammonium chloride, Quaternium-83, Quaternium-87, behenylamidopropyl-2,3-dihydroxypropyl-dimethylammonium chloride and palmitylamido-propyltrimethylammonium chloride.

6. The composition according to claim 1, wherein the cationic surfactants are present in an amount of 0.05% to 10% by weight relative to the total weight of the composition.

7. The composition according to claim 1, wherein the cationic polymer has a cationic charge density of 5 to 20 meq./g.

8. The composition according to claim 1, comprising at least one cationic polymer chosen from dialkyldiallylammonium halide homopolymers and copolymers, polyethyleneimines and polycondensates containing diquaternary ammonium or polyquaternary ammonium repeating units.

9. The composition according to claim 1, wherein the cationic polymer is present in a concentration of 0.01% to 10% by weight relative to the total weight of the composition.

10. The composition according to claim 1, comprising at least one starch chosen from corn starch, rice starch, cassava starch, barley starch, potato starch, wheat starch, sorghum starch and pea starch.

11. The composition according to claim 1, wherein the starch is chemically and/or physically modified.

12. The composition according to claim 1, comprising at least one starch modified via one or more of the following reactions: pregelatinization, oxidation, crosslinking, esterification, etherification, amidation, heat treatments.

13. The composition according to claim 1, comprising at least one starch modified via at least one esterification.

14. The composition according to claim 1, comprising at least one starch chosen from starch phosphates.

15. The composition according to claim 1, comprising at least one starch chosen from amphoteric starches.

16. The composition according to claim 15, wherein the amphoteric starch is a starch chloroethylamidodipropionate.

17. The composition according to claim 1, wherein the starch is present in a concentration of 0.01% to 10% by weight relative to the total weight of the composition.

18. The composition according to claim 1, comprising at least one solid compound chosen from oxyethylenated or non-oxyethylenated fatty alcohols, fatty esters, mineral waxes, and organic waxes other than fatty esters and fatty alcohols.

19. The composition according to claim 1, comprising at least one solid compound that is a plant or animal wax.

20. The composition according to claim 1, comprising at least one solid compound that is a linear fatty alcohol.

21. The composition according to claim 1, comprising at least one solid compound that is a saturated fatty alcohol.

22. The composition according to claim 1, comprising at least one solid compound that is a fatty alcohol having the structure R—OH, in which R denotes an optionally hydroxylated alkyl radical containing from 12 to 24 carbon atoms.

23. The composition according to claim 1, comprising at least one solid compound that is a fatty alcohol chosen from myristyl alcohol, cetyl alcohol, behenyl alcohol and stearyl alcohol.

24. The composition according to claim 18, comprising at least one fatty ester that is an ester of a carboxylic acid containing at least 10 carbon atoms and of a monoalcohol or a polyol.

25. The composition according to claim 24, wherein the fatty esters are chosen from esters of a monocarboxylic fatty acid containing at least 10 carbon atoms and of a monoalcohol containing at least 10 carbon atoms.

26. The composition according to claim 25, wherein the fatty esters are chosen from cetyl myristate and myristyl myristate.

27. The composition according to claim 18, comprising at least one wax chosen from beeswax, spermaceti, lanolin wax and lanolin derivatives; carnauba wax, candelilla wax, ouricury wax, Japan wax, cocoa butter, cork fibre wax or sugarcane wax; paraffin wax, petroleum jelly wax, lignite wax or microcrystalline waxes, ozokerites, olive wax, rice wax, hydrogenated jojoba wax or the absolute waxes of flowers, beeswaxes or modified beeswaxes (cerabellina), and natural or synthetic ceramides.

28. The composition according to claim 1, wherein the solid compound is present in a concentration of 0.1%-15% by weight relative to the total weight of the composition.

29. The composition according to claim 1, further comprising at least one additional conditioning agent.

30. The composition according to claim 29, wherein the at least one additional conditioning agent is chosen from silicones, cationic polymers other than cationic polymers with a cationic charge density of greater than or equal to 5 meq./g, liquid carboxylic esters, plant oils, mineral oils and synthetic oils, and mixtures thereof.

31. The composition according to claim 1, wherein the cosmetically acceptable aqueous medium comprises water and, optionally, at least one cosmetically acceptable solvent.

32. The composition according to claim 31, wherein the cosmetically acceptable aqueous medium comprises water and at least one cosmetically acceptable solvent chosen from C₁-C₄ lower alcohols, alkylene glycols, and C₅-C₁₀ alkanes.

33. The composition according to claim 1, further comprising at least one additive selected from anionic or amphoteric polymers, thickeners, opacifiers, nacreous agents, vitamins, provitamins, fragrances, dyes, organic or mineral particles, preserving agents and pH stabilizers.

34. The composition according to claim 1, wherein it is in the form of a hair conditioner, a composition for permanent-waving, relaxing, dyeing or bleaching the hair, or a rinse-out composition to be applied between the two steps of a permanent-waving or hair-relaxing operation.

35. The composition according to claim 1, wherein it is in the form of a rinse-out hair conditioner.

36. A process for treating keratin material, comprising applying the composition according to claim 1 to the keratin material, and then optionally rinsing.
